(19) United States
(12) Patent Application Publication
(10) Pub. No.: US 2017/0119289 A1
(43) Pub. Date: May 4, 2017

(54) FLUID COLLECTION DEVICE AND MEASUREMENT DEVICE

(71) Applicant: Seiko Epson Corporation, Tokyo (JP)

(72) Inventors: Satomi YOSHIOKA, Shiojiri (JP); Hiroshi YAGI, Tatsuno (JP)

(21) Appl. No.: 15/334,862

(22) Filed: Oct. 26, 2016

(30) Foreign Application Priority Data
Oct. 30, 2015 (JP) 2015-213923

(51) Int. Cl. A61B 5/145; A61B 5/1455

(52) U.S. Cl. CPC A61B 5/14517; A61B 5/14546; A61B 5/1455

(57) ABSTRACT

A sweat collection device as a fluid collection device includes a collection section which collects sweat, and a discharge section which discharges the sweat collected by the collection section from the collection section. Further, the sweat collection device is attached to the skin as an adherend, and the collection section is configured to be hermetically sealed by attaching the sweat collection device to the skin. Further, the sweat collection device includes a transfer section which connects the collection section and the discharge section to each other. Further, the sweat collection device includes a gel-like response section which is placed on any of the collection section, the transfer section, and the discharge section, and responds to lactic acid contained in sweat.

This application claims the benefit of Japanese Patent Application No. 2015-213923, filed on Oct. 30, 2015. The content of the aforementioned application is hereby incorporated by reference in its entirety.

### BACKGROUND

1. Technical Field

The present invention relates to a fluid collection device and a measurement device including the fluid collection device.

2. Related Art

In the past, as a method for obtaining biological information in the body, a biochemical test for examining the composition of blood obtained by blood collection has been generally performed. This test is mainly performed in medical institutions. Above all, a blood glucose sensor has been widely used in diabetic patients, and also a simple lactic acid sensor is getting widely used in athletes. However, these sensors are both test methods involving blood collection using an invasive technique. In order to perform health management easily in a daily life using a sensor by ordinary people including athletes, etc., a sensor employing a non-invasive technique for obtaining biological information in the body using a component excreted outside the body is needed.

As one example of the sensor employing a non-invasive technique, JP-A-2009-247440 (PTL 1) discloses a sweat recovery device, in which sweating is promoted by a sweating promotion section, and a sweat recovery section is moved to a place where sweating is promoted to recover excreted sweat, and a sweat analyzer which analyzes recovered sweat.

However, In PTL 1, the sweat recovery device involves the mechanical movement and therefore has a problem that the configuration is complicated. In addition, a configuration of discharging sweat recovered by the sweat recovery device is not disclosed, and therefore, it is considered that the recovered sweat is retained in the sweat recovery device, and therefore, the technique has a problem that it is difficult to continuously recover and analyze sweat.

Accordingly, a fluid collection device capable of continuously recovering (collecting) a fluid (liquid) excreted outside the body including sweat and also capable of continuously measuring a specific component in the fluid using the collected fluid with a simple configuration, and a measurement device including the fluid collection device have been demanded.

### SUMMARY

An advantage of some aspects of the invention is to solve at least part of the problems described above and the invention can be implemented as the following modes or application examples.

### APPLICATION EXAMPLE 1

A fluid collection device according to this application example includes a collection section which collects a fluid, and a discharge section which discharges the fluid collected by the collection section from the collection section.

According to this application example, the fluid collection device includes a discharge section, and therefore, discharges the fluid collected by the collection section. Due to this, the fluid (fluid excreted outside the body) collected by the collection section can be prevented from being retained in the collection section, and thus, the fluid can be continuously collected.

### APPLICATION EXAMPLE 2

In the fluid collection device according to the above application example, it is preferred that the fluid collection device is attached to an adherend, and the collection section is configured to be hermetically sealed by attaching the fluid collection device to the adherend.

According to such a fluid collection device, the collection section is hermetically sealed by attaching the fluid collection device to the adherend, and therefore, the fluid can be collected by the collection section without leakage. Due to this, the fluid collected by the collection section can be made to flow smoothly from the collection section to the discharge section by the pressure of the fluid to be sequentially collected.

### APPLICATION EXAMPLE 3

In the fluid collection device according to the above application example, it is preferred that the fluid collection device includes a transfer section which connects the collection section and the discharge section to each other.

According to such a fluid collection device, the fluid collection device includes a transfer section which connects the collection section and the discharge section to each other and transfers the fluid, and therefore, the transfer of the fluid from the collection section to the discharge section can be assisted. Further, it is also possible to transfer the fluid accumulated in the collection section to the discharge section all at once.

### APPLICATION EXAMPLE 4

In the fluid collection device according to the above application example, it is preferred that the fluid collection device includes a gel-like response section which is placed on any of the collection section, the transfer section, and the discharge section, and responds to a predetermined component contained in the fluid.

According to such a fluid collection device, a gel-like response section which responds to a predetermined component contained in the collected fluid is placed on any of the collection section, the transfer section, and the discharge section, and therefore, by using the collected fluid, the specific component in the fluid can be measured. In addition, since the collected fluid continuously flows from the collection section to the discharge section, the specific component in the fluid can be continuously measured.

### APPLICATION EXAMPLE 5

In the fluid collection device according to the above application example, it is preferred that any of the collection section, the transfer section, and the discharge section, on which the response section is placed, includes a region capable of visually recognizing the response section which responds to the predetermined component contained in the fluid.

According to such a fluid collection device, a region capable of confirming the color of the response section is included, and therefore, a user can confirm the measured amount of the specific component in the fluid by the color, and also can confirm the change (increase or decrease) in the amount of the specific component by the change in the color (color tone).

### APPLICATION EXAMPLE 6

In the fluid collection device according to the above application example, it is preferred that the collection section includes a setting section to be set on the adherend, and the setting section and the response section are spaced apart from each other.

According to such a fluid collection device, a space region is provided between the setting section and the response section in the thickness direction (these members are spaced apart from each other), and therefore, the fluid collected from the adherend is accumulated in this space region, and thus can be made to react with the response section. In addition, by this space region, the fluidity of the fluid is improved as compared with the case where the response section is in contact with the adherend without any gaps. Due to this, the response section can come into contact with the continuously collected fluid at all times, and therefore, the response section can responds according to the change in the amount of the specific component in the fluid to be collected. As a result, the responsiveness of the response section can be improved.

### APPLICATION EXAMPLE 7

In the fluid collection device according to the above application example, it is preferred that the surfaces coming into contact with the fluid of the collection section, the transfer section, and the discharge section have hydrophilicity.

According to such a fluid collection device, the fluidity of the collected fluid in the collection section, the transfer section, and the discharge section can be improved.

### APPLICATION EXAMPLE 8

In the fluid collection device according to the above application example, it is preferred that the discharge section is configured to include a hole member constituted by a plurality of holes each of which is continuous with the outside of the fluid collection device from the collection section or the transfer section.

According to such a fluid collection device, the discharge section is configured to include a hole member, and therefore, the fluid collected by the collection section can be efficiently discharged to the outside by utilizing a force of sucking the fluid (for example, a capillary force) of the hole member.

### APPLICATION EXAMPLE 9

In the fluid collection device according to the above application example, it is preferred that the fluid collection device includes a plurality of discharge sections in an outer peripheral portion of the collection section.

According to such a fluid collection device, the fluid collected by the collection section can be made to more efficiently flow to the discharge sections and can be more efficiently discharged from the discharge sections to the outside.

### APPLICATION EXAMPLE 10

In the fluid collection device according to the above application example, it is preferred that the discharge section is configured to be continuous along an outer peripheral portion of the collection section.

According to such a fluid collection device, the discharge section is configured to be continuous along an outer peripheral portion of the collection section, and therefore, the fluid collected by the collection section can be made to more efficiently flow to the discharge section and can be more efficiently discharged from the discharge section to the outside.

### APPLICATION EXAMPLE 11

In the fluid collection device according to the above application example, it is preferred that the collection section includes an accumulation section which surrounds the response section and accumulates the fluid.

According to such a fluid collection device, the collection section includes an accumulation section which surrounds the response section and accumulates the fluid, and therefore, the response section can be made to reliably respond to the fluid.

### APPLICATION EXAMPLE 12

In the fluid collection device according to the above application example, it is preferred that the response section is configured to include a stimulus-responsive gel whose volume expands in response to the fluid.

According to such a fluid collection device, in the case where fine particles with high reflectance are incorporated in the response section, by the expansion of the volume of the stimulus-responsive gel, the interparticle spacing changes, so that the wavelength of the reflection light to be reflected changes, and thus, the color changes. According to this, the amount of the specific component in the fluid can be easily measured as the change in the color. In addition, by the expansion of the volume of the stimulus-responsive gel, the electrical conductivity of the stimulus-responsive gel changes. Therefore, by measuring the electrical conductivity of the response section, the amount of the specific component in the fluid can be easily measured as the change in the electrical conductivity.

### APPLICATION EXAMPLE 13

In the fluid collection device according to the above application example, it is preferred that the fluid collection device includes an adhesive member for attaching the collection section to the adherend.

According to such a fluid collection device, the collection section is attached to the adherend by the adhesive member, and therefore, the collection section can be easily brought into close contact with the adherend.

### APPLICATION EXAMPLE 14

A measurement device according to this application example includes the fluid collection device according to any of the above application examples, a conversion section which converts a change in a response section into a data signal and outputs the data signal, a calculation section which calculates the amount of a predetermined component contained in a fluid from the data signal output by the conversion section, a display section which displays the result of calculation by the calculation section, and a housing section which houses the response section, the conversion section, the calculation section, and the display section.

According to such a measurement device, a change in the response section of the fluid collection device is converted into a data signal by the conversion section, and the amount of a predetermined component contained in a fluid is calculated by the calculation section from the data signal output by the conversion section. Further, the result of calculation by the calculation section is displayed on the display section. Incidentally, the response section, the conversion section, the calculation section, and the display section are housed in the housing section. The measurement device includes the fluid collection device which continuously collects a fluid and responds to the collected fluid as described above, and therefore, can continuously measure the amount of a specific component in the fluid using a non-invasive technique, and can display the measurement. Accordingly, by attaching this measurement device to an adherend (for example, an arm or the like), a user can easily confirm the change in the amount of a specific component in the fluid.

### BRIEF DESCRIPTION OF THE DRAWINGS

The invention will be described with reference to the accompanying drawings, wherein like numbers reference like elements.

FIG. 1 is a plan view schematically showing a configuration of a sweat collection device according to a first embodiment.

FIG. 2 is a cross-sectional view schematically showing a configuration of the sweat collection device according to the first embodiment.

FIG. 3 is a plan view schematically showing a configuration of a sweat collection device according to a second embodiment.

FIG. 4 is a cross-sectional view schematically showing a configuration of the sweat collection device according to the second embodiment.

FIG. 5 is a plan view schematically showing a configuration of a sweat collection device according to a third embodiment.

FIG. 6 is a cross-sectional view schematically showing a configuration of the sweat collection device according to the third embodiment.

FIG. 7 is a plan view schematically showing a configuration of a sweat collection device according to a fourth embodiment.

FIG. 8 is a cross-sectional view schematically showing a configuration of the sweat collection device according to the fourth embodiment.

FIG. 9 is a plan view schematically showing a configuration of a sweat collection device according to a fifth embodiment.

FIG. 10 is a cross-sectional view schematically showing a configuration of the sweat collection device according to the fifth embodiment.

FIG. 11 is a cross-sectional view schematically showing a configuration of a sweat measurement device according to a sixth embodiment.

FIG. 12 is a view showing a schematic circuit configuration of the sweat measurement device according to the sixth embodiment.

FIG. 13 is a cross-sectional view schematically showing a configuration of a sweat measurement device according to a seventh embodiment.

FIG. 14 is a view showing a schematic circuit configuration of the sweat measurement device according to the seventh embodiment.

### DESCRIPTION OF EXEMPLARY EMBODIMENTS

Hereinafter, embodiments will be described with reference to the accompanying drawings. Incidentally, the respective members in the respective drawings are shown by changing the scale for each member so as to have a recognizable size in the respective drawings.

### First Embodiment

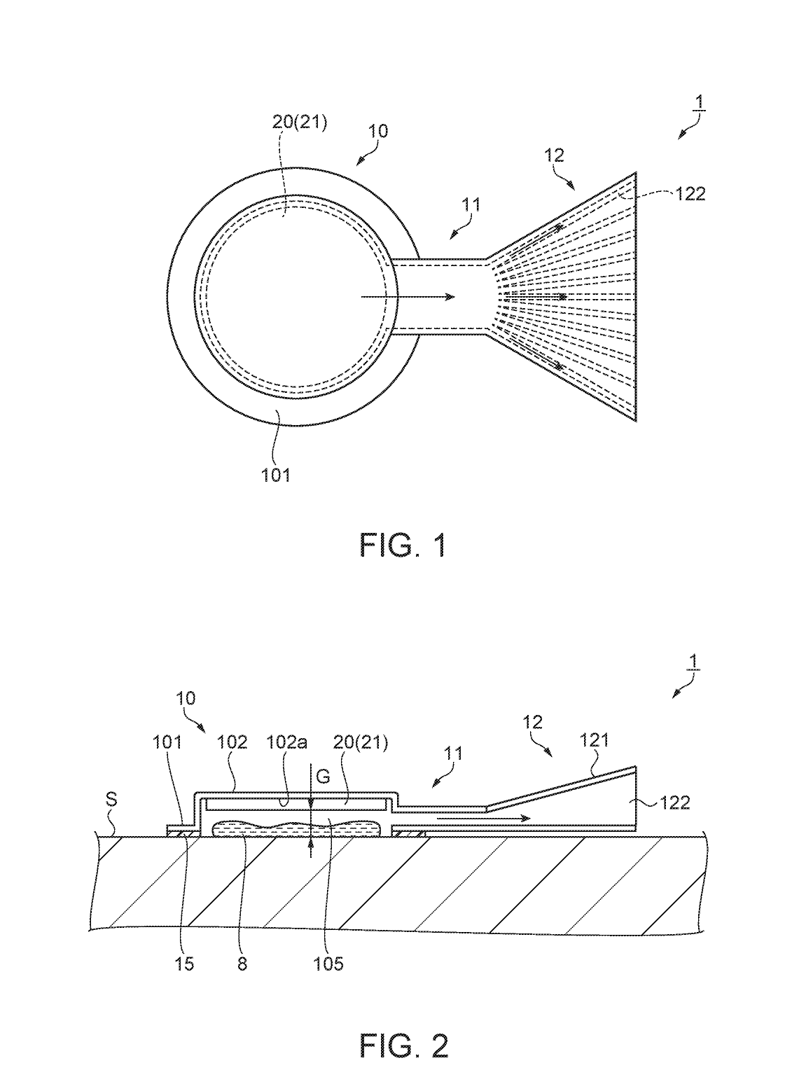

FIG. 1 is a plan view schematically showing a configuration of a sweat collection device 1 according to a first embodiment. FIG. 2 is a cross-sectional view schematically showing a configuration of the sweat collection device 1. In FIG. 1, the inside of the sweat collection device 1 is indicated by a broken line. The configuration of the sweat collection device 1 as a fluid collection device will be described with reference to FIGS. 1 and 2.

The sweat collection device 1 of this embodiment is a device to be used by being attached to the human body surface, for example, the arm surface as an adherend. Further, the sweat collection device 1 collects excreted sweat 8 as a fluid (liquid) excreted outside the body, and measures the amount of lactic acid as a specific component contained in the sweat 8. A user can confirm the amount of lactic acid at any time by attaching the sweat collection device 1 to the arm or the like and performing an exercise such as jogging.

As shown in FIGS. 1 and 2, the sweat collection device 1 is configured to include a collection section 10, a transfer section 11, a discharge section 12, and a response section 20. Further, the sweat collection device 1 includes a sheet-shaped adhesive member 15 for adhering the collection section 10 to the surface of the skin S. The sweat collection device 1 is configured such that the collection section 10, the transfer section 11, and the discharge section 12 are integrally formed.

Incidentally, hereinafter, for the sake of convenience of explanation, in the sweat collection device 1, the normal direction on the surface of the skin S is referred to as “upper direction”, and the opposite direction thereto is referred to as “lower direction”.

In this embodiment, in the sweat collection device 1, the entire outer case is formed from a transparent synthetic resin material, and the state of the inside can be confirmed (can be visually recognized). As the material of the sweat collection device 1, for example, a transparent acrylic resin, a transparent epoxy resin, a transparent ABS (acrylonitrile-butadiene-styrene copolymer) resin, a transparent PC (polycarbonate) resin, or the like can be used. However, the material is not limited thereto, and a soft glass or the like can also be used.

The collection section 10 is formed into a concave cylindrical shape in which the bottom surface is opened and the upper surface is closed. Further, a bottom surface portion is provided with a setting section 101 formed into a doughnut-like flat plate shape extending from the outer periphery to the outside. The adhesive member 15 is placed on the lower surface of the setting section 101, and by adhering the collection section 10 to the skin S through the adhesive member 15, the sweat collection device 1 can be attached to the skin S. The collection section 10 of the sweat collection device 1 is configured to be hermetically sealed by attaching the sweat collection device 1 to the skin S.

The transfer section 11 is configured to be connected to (continuous with) the collection section 10. The transfer section 11 extends from the side surface of the outer periphery of the collection section 10 to the outside and is formed into a cylindrical shape with a flat rectangular cross section. Then, the discharge section 12 is configured to be connected to (continuous with) the transfer section 11. The discharge section 12 is formed radiating from the terminal end of the transfer section 11.

On an inner surface 102a of an upper surface 102 of the collection section 10, the response section 20 formed into a circular plate shape is placed. The response section 20 is configured to include a stimulus-responsive gel 21. The stimulus-responsive gel 21 is a gel-like material containing an agent which responds to sweat. The constituent material of the stimulus-responsive gel 21 is not particularly limited, however, in this embodiment, it is constituted by, for example, a material containing a polymer material having a crosslinked structure and a solvent, or the like, and the polymer material serves as the agent which responds to sweat.

As the polymer material constituting the stimulus-responsive gel 21, for example, a material obtained by reacting a monomer, a polymerization initiator, a crosslinking agent, or the like can be used. Incidentally, the term “tanryotai (in Japanese)” is also referred to as “monomer”.

Examples of the monomer include acrylamide, N-methylacrylamide, N-isopropylacrylamide, N,N-dimethylacrylamide, N,N-dimethylaminopropylacrylamide, various quaternary salts of N,N-dimethylaminopropylacrylamide, acryloylmorpholine, various quaternary salts of N,N-dimethylaminoethylacrylate, acrylic acid, various alkyl acrylates, methacrylic acid, various alkyl methacrylates, 2-hydroxyethylmethacrylate, glycerol monomethacrylate, N-vinylpyrrolidone, acrylonitrile, styrene, polyethylene glycol diacrylate, 1,6-hexanediol diacrylate, neopentyl glycol diacrylate, tripropylene glycol diacrylate, polypropylene glycol diacrylate, 2,2-bis[4-(acryloxydiethoxy)phenyl]propane, 2,2-bis[4-(acryloxypolyethoxy)phenyl]propane, 2-hydroxy-1-acryloxy-3-methacryloxypropane, 2,2-bis[4-(acryloxypolypropoxy)phenyl]propane, ethylene glycol dimethacrylate, diethylene glycol dimethacrylate, triethylene glycol dimethacrylate, polyethylene glycol dimethacrylate, 1,3-butylene glycol dimethacrylate, 1,6-hexanediol dimethacrylate, neopentyl glycol dimethacrylate, polypropylene glycol dimethacrylate, 2-hydroxy-1,3-dimethacryloxypropane, 2,2-bis[4-(methacryloxyethoxy)phenyl]propane, 2,2-bis[4-(methacryloxyethoxydiethoxy)phenyl]propane, 2,2-bis[4-(methacryloxyethoxypolyethoxy)phenyl]propane, trimethylolpropane trimethacrylate, tetramethylolmethane trimethacrylate, trimethylolpropane triacrylate, tetramethylolmethane triacrylate, tetramethylolmethane tetraacrylate, dipentaerythritol hexaacrylate, N,N′-methylenebisacrylamide, N,N′-methylenebismethacrylamide, diethylene glycol diallyl ether, and divinylbenzene.

As the monomer for detecting lactic acid, 3-acrylamidophenylboronic acid, vinylphenylboronic acid, acryloyloxyphenylboronic acid, N-isopropylacrylamide (NIPAAm), ethylenebisacrylamide, N-hydroxyethylacrylamide, or the like can be preferably used. More specifically, it is preferred to use at least one monomer selected from the group consisting of 3-acrylamidophenylboronic acid, vinylphenylboronic acid, and acryloyloxyphenylboronic acid, and at least one monomer selected from the group consisting of N-isopropylacrylamide (NIPAAm), ethylenebisacrylamide, and N-hydroxyethylacrylamide in combination as the monomer.

The polymerization initiator can be appropriately selected according to, for example, the polymerization form thereof. More specifically, as the polymerization initiator, a compound which generates radicals by ultraviolet light such as hydrogen peroxide, a persulfate such as potassium persulfate, sodium persulfate, or ammonium persulfate, an azo-based initiator such as 2,2′-azobis(2-amidinopropane) dihydrochloride, 2,2′-azobis(N,N′-dimethyleneisobutylamidine) dihydrochloride, 2,2′-azobis{2-methyl-N-[1,1-bis(hydroxymethyl)-2-hydroxyethyl]propionamide}, 2,2′-azobis[2-(2-imidazolin-2-yl)propane] dihydrochloride, 4,4′-azobis(4-cyanovaleric acid), 2,2′-azobisisobutyronitrile, 2,2′-azobis(2,4′-dimethylvaleronitrile), benzophenone, 2,2-dimethoxy-1,2-diphenylethan-1-one, 1-hydroxycyclohexyl phenyl ketone, 2-hydroxy-2-methyl-1-phenylpropan-1-one, 2,4,6-trimethylbenzoyl diphenylphosphine oxide, or 1-[4-(2-hydroxyethoxy)-phenyl]-2-hydroxy-2-methyl-1-propan-1-one, a compound which generates radicals by light with a wavelength of 360 nm or more such as a substance obtained by mixing a thiopyrylium salt-based, merocyanine-based, quinoline-based, or styrylquinoline-based dye with 2,4-diethyl thioxanthone, isopropyl thioxanthone, 1-chloro-4-propoxythioxanthone, 2-(3-dimethylamino-2-hydroxypropoxy)-3,4-dimethyl-9H-thiox anthon-9-one methochloride, 2-methyl-1[4-(methylthio)phenyl]-2-morpholinopropane-1,2-b enzyl-2-dimethylamino-1-(4-morpholinophenyl)-butanone-1, bis(cyclopentadienyl)-bis(2,6-difluoro-3-(pyl-1-yl) titanium, or a peroxy ester such as 1,3-di(t-butylperoxycarbonyl)benzene or 3,3′,4,4′-tetra-(t-butylperoxycarbonyl)benzophenone, or the like can be used. In addition, hydrogen peroxide or a persulfate can also be used as a redox-based initiator in combination with, for example, a reducing substance such as a sulfite or L-ascorbic acid, an amine salt, or the like.

As the crosslinking agent, a compound having two or more polymerizable functional groups can be used, and more specifically, ethylene glycol, propylene glycol, trimethylolpropane, glycerin, polyoxyethylene glycol, polyoxypropylene glycol, polyglycerin, N,N′-methylenebisacrylamide, N,N-methylene-bis-N-vinylacetamide, N,N-butylene-bis-N-vinylacetamide, tolylene diisocyanate, hexamethylene diisocyanate, allylated starch, allylated cellulose, diallyl phthalate, tetraallyloxyethane, pentaerythritol triallyl ether, trimethylolpropane triallyl ether, diethylene glycol diallyl ether, triallyl trimellitate, or the like can be used.

The stimulus-responsive gel 21 may contain a plurality of different types of polymer materials. The content of the polymer material in the stimulus-responsive gel 21 is preferably 0.7 mass % or more and 36.0 mass % or less, more preferably 2.4 mass % or more and 27.0 mass % or less.

When the stimulus-responsive gel 21 is configured to include a solvent, the polymer material can be favorably gelled. As the solvent, any of various types of organic solvents and inorganic solvents can be used. Specific examples of the solvent include water; various types of alcohols such as methanol and ethanol; ketones such as acetone; ethers such as tetrahydrofuran and diethyl ether; amides such as dimethylformamide; chain aliphatic hydrocarbons such as n-pentane, n-hexane, n-heptane, and n-octane; alicyclic hydrocarbons such as cyclohexane and methylcyclohexane; and aromatic hydrocarbons such as benzene, toluene, and xylene, however, in particular, a solvent containing water is preferred.

The stimulus-responsive gel 21 may be configured to contain a plurality of different types of components as the solvent. The content of the solvent in the stimulus-responsive gel 21 is preferably 30 mass % or more and 95 mass % or less, more preferably 50 mass % or more and 90 mass % or less.

The size of the stimulus-responsive gel 21 is not particularly limited, however, in this embodiment, for example, the plane size of the stimulus-responsive gel 21 is set to about 1 cm. Further, the thickness of the stimulus-responsive gel 21 is preferably 1 μm or more and 1 mm or less, more preferably 5 μm or more and 500 μm or less.

The stimulus-responsive gel 21 has a polymer chain. In the polymer chain, many boronic acid groups are included. When the stimulus-responsive gel 21 is in a state where lactic acid does not permeate the gel (the gel does not respond to lactic acid), the stimulus-responsive gel 21 is in a state where the boronic acid groups are bound to each other so that the polymer chains come close to each other. Due to this, the stimulus-responsive gel 21 is in a contracted state. When the stimulus-responsive gel 21 is in a state where lactic acid permeates the gel (the gel responds to lactic acid), the stimulus-responsive gel 21 is brought to a state where the boronic acid group and lactic acid are bound to each other so that the polymer chains are dissociated from each other. As a result, the volume of the stimulus-responsive gel 21 expands.

In this embodiment, the stimulus-responsive gel 21 is configured to further include fine particles with high reflectance in addition to the polymer material and the solvent. According to this, in the stimulus-responsive gel 21, when the lattice spacing of colloidal crystals is near the visible light range, the colloidal crystal emits a structural color characteristic of the interparticle spacing, and when lactic acid is incorporated in the gel, the structural color changes due to the change in the interparticle spacing. In other words, the stimulus-responsive gel 21 emits reflection light with a wavelength characteristic of the interparticle spacing, and the wavelength of the reflection light to be reflected changes due to the change in the interparticle spacing by the expansion of the volume.

The stimulus-responsive gel 21 emits a color with a short wavelength when the lactic acid level is low and emits a color with a longer wavelength as the lactic acid level increases. More specifically, in this embodiment, the stimulus-responsive gel 21 emits a blue color when the lactic acid level is low or lactic acid is not present, and as the lactic acid level increases, the color changes to green, yellow, and red. When the lactic acid level decreases, the order of the change in the color is reversed. A user can easily visually recognize the change in the color of the stimulus-responsive gel 21 through the upper surface 102 of the collection section 10 configured to be transparent.

Examples of the constituent material of the fine particles include inorganic materials such as silica and titanium oxide; and organic materials such as polystyrene, polyester, polyimide, polyolefin, poly(methyl (meth)acrylate), polyethylene, polypropylene, polyether sulfone, nylon, polyurethane, polyvinyl chloride, and polyvinylidene chloride. The fine particles are preferably silica fine particles. According to this, the shape stability and the like of the fine particles are made particularly excellent, and the durability, reliability, and the like of the stimulus-responsive gel can be made particularly excellent. In addition, the silica fine particles are relatively easily available as monodispersed fine particles having a sharp particle size distribution, and therefore are advantageous also from the viewpoint of stable production and supply of the stimulus-responsive gel.

The shape of the fine particle is not particularly limited, but is preferably a spherical shape. According to this, the structural color due to the colloidal crystals or the change in the structural color is easily visually recognized, and therefore, the detection of a specific component can be more easily performed. The average particle diameter of the fine particles is not particularly limited, but is preferably 10 nm or more and 1000 nm or less, more preferably 20 nm or more and 500 nm or less.

According to this, when a specific component is incorporated in the stimulus-responsive gel, the structural color due to the colloidal crystals or the change in the structural color is more easily visually recognized, and therefore, the detection of a specific component can be more easily performed. In addition, since the structural color due to the colloidal crystals is more easily visually recognized, the quantitative determination of a specific component can also be more easily and accurately performed by the color tone.

As the average particle diameter, the average particle diameter on a volume basis is shown. For example, the measurement is performed using a particle size distribution analyzer employing a Coulter counter method for a dispersion liquid obtained by adding a sample to methanol and dispersing the sample therein for 3 minutes with an ultrasonic disperser. At this time, the average particle diameter of the sample can be obtained by performing the measurement using an aperture of 50 μm.

The stimulus-responsive gel 21 may contain a plurality of different types of fine particles. The content of the fine particles in the stimulus-responsive gel 21 is preferably 1.6 mass % or more and 36 mass % or less, more preferably 4.0 mass % or more and 24 mass % or less.

As shown in FIG. 2, in order to place the stimulus-responsive gel 21 on the inner surface 102a of the upper surface 102 of the collection section 10, in this embodiment, first, the inner surface 102a of the collection section 10, on which the stimulus-responsive gel 21 is to be placed, is subjected to an oxygen plasma treatment or the like, whereby the wettability of the surface is improved. Thereafter, the polymer material, the solvent, and the fine particles constituting the stimulus-responsive gel 21 are weighed, respectively, and mixed uniformly, and the resulting liquid is applied to the inner surface 102a of the collection section 10. Then, the liquid is irradiated with ultraviolet light, whereby the stimulus-responsive gel 21 is fixed to the inner surface 102a. The fixing may also be performed using heat other than ultraviolet light.

In the case where the response section 20 (stimulus-responsive gel 21) is placed on the collection section 10, a space region 105 to serve as a gap G is formed between the setting section 101 (specifically, the adhesive member 15) to be set on the skin S and the response section 20 in the thickness direction. In other words, the setting section 101 and the response section 20 are spaced apart from each other. Incidentally, the space region 105 serves as an accumulation region for accumulating the sweat 8 excreted from the skin S in the collection section 10. Further, the space region 105 improves the fluidity of the sweat 8 as compared with the case where the response section 20 is in contact with the skin S without any gaps.

When the sweat collection device 1 is attached to the surface of the skin S, the gap G between the skin S and the response section 20 (stimulus-responsive gel 21) is not particularly limited, but is preferably, for example, 1 μm or more and 1 mm or less, more preferably 5 μm or more and 500 μm or less in this embodiment. In addition, it is preferred that the gap G is larger than the thickness of the response section 20 (stimulus-responsive gel 21).

By setting the thickness of the stimulus-responsive gel 21 to be smaller (thinner) than the gap G, even a very small amount of the sweat 8 permeates the entire inside of the stimulus-responsive gel 21, and the volume of the entire stimulus-responsive gel 21 changes. As a result, the newly excreted sweat 8 permeates the entire inside of the stimulus-responsive gel 21 at once, and thus, the immediate measurement can be performed in response to the excreted sweat 8.

The discharge section 12 is constituted by a fine communication hole member (also simply referred to as “hole member”). More specifically, in the discharge section 12, many (a plurality of) hole sections 122 (capillaries) are formed by covering grooves formed into narrow slits to become fine communication holes from the terminal end of the transfer section 11 (a start end portion of the discharge section 12) to a terminal end portion of the discharge section 12 with a cover 121 from the upper side. Each of these hole sections 122 is opened at the start end portion and the terminal end portion of the discharge section 12, and has a function to suck the sweat in the transfer section 11 by a capillary force and to discharge the sweat to the outside air (the outside of the sweat collection device). In other words, the transfer section 11 is connected to the outside air through the holes of the hole member.

Incidentally, the inner surface to come into contact with the sweat 8 of each of the collection section 10, the transfer section 11, and the discharge section 12 constituting the sweat collection device 1 has hydrophilicity by being subjected to a hydrophilic treatment. According to this, the sweat 8 is easily transferred on the inner surface of each of the members. The hydrophilic treatment is not particularly limited, and various methods can be used. For example, in this embodiment, each inner surface to come into contact with the sweat 8 is subjected to an oxygen plasma treatment. The hydrophilic treatment may be performed by applying a solution containing a silane coupling agent.

The operation of the sweat collection device 1 will be described.

When the sweat collection device 1 is attached to an arm surface (skin S) and an exercise is performed, the sweat 8 excreted from the skin S is accumulated in the space region 105 of the collection section 10. The sweat 8 accumulated in the space region 105 comes into contact with the response section 20 (stimulus-responsive gel 21) placed on the upper part thereof and permeates the inside of the stimulus-responsive gel 21. Then, the stimulus-responsive gel 21 absorbs the sweat 8 and responds to (reacts with) lactic acid contained in the sweat 8. The volume of the stimulus-responsive gel 21 expands depending on the amount having reacted with lactic acid.

In this embodiment, silica fine particles are incorporated in the stimulus-responsive gel 21, and a substantially blue color is emitted in a state where the gel does not react with lactic acid. Since the sweat collection device 1 is transparent, a user can visually recognize the blue color emitted by the stimulus-responsive gel 21 through the upper surface 102 of the collection section 10.

By attaching the collection section 10 to the skin S, the device is in a hermetically sealed configuration except for the transfer section 11, and therefore, in the case where sweating continues and the sweat 8 is accumulated in the space region 105, due to the pressure of the excreted sweat 8, the sweat 8 is transferred to the direction of releasing the pressure. More specifically, when the inside of the collection section 10 is filled with the sweat 8, the sweat 8 dissolved in the stimulus-responsive gel 21 or the sweat 8 accumulated in the space is pushed out to the transfer section 11 located on the side where the pressure is released.

The sweat 8 transferred to the transfer section 11 flows inside the transfer section 11 and reaches the discharge section 12 connected to the transfer section 11. The sweat 8 having reached the discharge section 12 flows inside the plurality of hole sections 122 by the pressure of the sweat 8 and a capillary force of the plurality of hole sections 122 and reaches the terminal end portion of the discharge section 12, and then, is discharged to the outside air from the discharge section 12. Incidentally, in FIGS. 1 and 2, the flow of the sweat 8 is schematically shown by the arrow.

As described above, when sweating continues, the collected sweat 8 sequentially flows from the collection section 10 to the discharge section 12 by the pressure of the sweat 8. Due to this, the collected sweat 8 is not retained in the collection section 10, and smoothly flows to the transfer section 11 and to the discharge section 12. Therefore, the sweat collection device 1 can continuously collect the excreted sweat 8. Further, the stimulus-responsive gel 21 can come in contact with the continuously collected sweat 8 at all times, and thus can react with lactic acid in the amount contained in the collected sweat 8 immediately after sweating. Then, the stimulus-responsive gel 21 can continuously measure the result of the reaction as a change in the color. In addition, the sweat 8 after measurement is promptly transferred from the stimulus-responsive gel 21.

The volume of the stimulus-responsive gel 21 expands as the amount of lactic acid contained in the collected sweat 8 increases, and the color emitted by the stimulus-responsive gel 21 changes. As described above, the stimulus-responsive gel 21 of this embodiment is configured such that it emits a blue color in the case where it does not react with lactic acid, and when it starts to react with lactic acid and the amount of lactic acid increases, the color changes to red. Therefore, a user can visually perceive the amount of lactic acid by confirming the change in the color (color tone) of the stimulus-responsive gel 21 at any time during an exercise.

According to the above-mentioned embodiment, the following advantageous effects are obtained.

The sweat collection device 1 as the fluid collection device of this embodiment includes the discharge section 12, and therefore, discharges the sweat 8 collected by the collection section 10. Due to this, the sweat 8 collected by the collection section 10 can be prevented from being retained in the collection section 10, and thus, the sweat 8 can be continuously collected.

According to the sweat collection device 1 of this embodiment, the collection section 10 is hermetically sealed by attaching the sweat collection device 1 to the skin S, and therefore, the sweat 8 can be collected by the collection section 10 without leakage. Due to this, the sweat 8 collected by the collection section 10 can be made to flow smoothly from the collection section 10 to the discharge section 12 by the pressure of the sweat 8 to be sequentially collected.

According to the sweat collection device 1 of this embodiment, the sweat collection device 1 includes the transfer section 11 which transfers the sweat 8 between the collection section 10 and the discharge section 12, and therefore, the transfer of the sweat 8 from the collection section 10 to the discharge section 12 can be assisted. Further, since the volume of the transfer section 11 is smaller than the volume of the collection section 10, it is also possible to transfer the sweat 8 accumulated in the collection section 10 to the discharge section 12 all at once.

According to the sweat collection device 1 of this embodiment, the response section 20 is placed on the collection section 10 and is configured to include the stimulus-responsive gel 21 whose volume expands in response to lactic acid contained in the sweat 8. Therefore, by using the collected sweat 8, lactic acid as the specific component contained in the sweat 8 can be measured. In addition, since the collected sweat 8 continuously flows from the collection section 10 to the discharge section 12, lactic acid contained in the sweat 8 can be continuously measured. In addition, fine particles with high reflectance are incorporated in the stimulus-responsive gel 21, and therefore, by the expansion of the volume of the stimulus-responsive gel 21, the interparticle spacing changes, so that the wavelength of the reflection light to be reflected changes, and thus, the color changes. According to this, the amount of lactic acid contained in the sweat 8 can be easily measured as the change in the color.

According to the sweat collection device 1 of this embodiment, the collection section 10, on which the response section 20 is placed, is formed from a transparent synthetic resin. Therefore, a user can confirm the amount of lactic acid contained in the sweat 8 by the color of the stimulus-responsive gel 21 through the transparent collection section 10, and also can confirm the change (increase or decrease) in the amount of lactic acid as the change in the color (color tone).

According to the sweat collection device 1 of this embodiment, the space region 105 is provided between the setting section 101 of the collection section 10 and the response section 20 in the thickness direction. Therefore, the sweat 8 collected from the skin S is accumulated in this space region 105, and thus can be made to react with the response section 20. In addition, by this space region 105, the fluidity of the sweat 8 is improved as compared with the case where the response section 20 is in contact with the skin S without any gaps.

According to the sweat collection device 1 of this embodiment, by the collection section 10 (including the space region 105), the transfer section 11, and the discharge section 12, the fluidity of the sweat 8 can be improved, and therefore, the response section 20 can come into contact with the continuously collected sweat 8 at all times, and thus, the response section 20 can responds according to the change in the amount of lactic acid contained in the sweat 8 to be collected. As a result, the responsiveness of the response section 20 can be improved.

According to the sweat collection device 1 of this embodiment, the inner surfaces coming into contact with the sweat 8 of the collection section 10, the transfer section 11, and the discharge section 12 have hydrophilicity. Therefore, the fluidity of the collected sweat 8 in the collection section 10, the transfer section 11, and the discharge section 12 can be improved.

According to the sweat collection device 1 of this embodiment, the discharge section 12 is configured to include the hole sections 122 as fine communication holes. Therefore, the sweat 8 collected by the collection section 10 can be efficiently discharged to the outside by utilizing a force of sucking the sweat 8 (a capillary force) of the fine communication holes.

According to the sweat collection device 1 of this embodiment, the sweat collection device includes the adhesive member 15 for attaching the collection section 10 to the skin S. Therefore, by attaching the collection section 10 to the skin S, the collection section 10 can be easily brought into close contact with the skin S.

According to the sweat collection device 1 of this embodiment, the sweat collection device 1 is a sensor using not blood, but sweat 8 as an analyte, and therefore, the information of biological metabolism can be easily obtained non-invasively without collecting blood. Due to this, the use of the sweat collection device 1 in a daily life is advantageous for performing health management for each individual or giving training advice to each individual.

### Second Embodiment

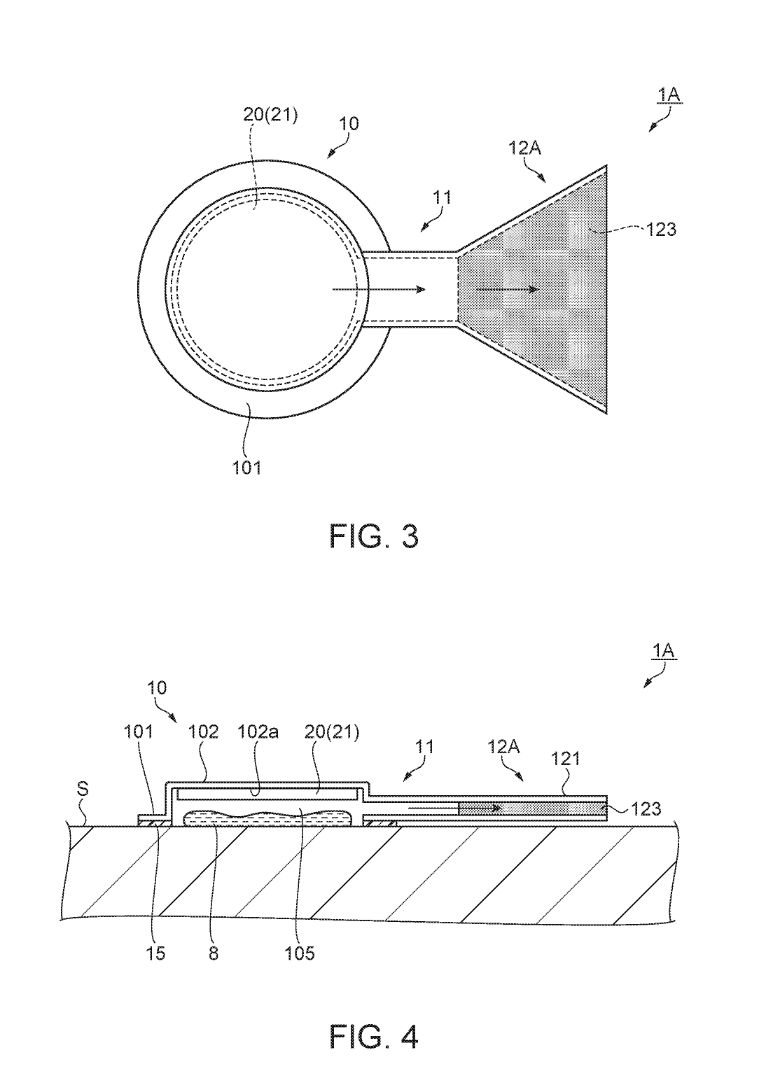

FIG. 3 is a plan view schematically showing a configuration of a sweat collection device 1A according to a second embodiment. FIG. 4 is a cross-sectional view schematically showing a configuration of the sweat collection device 1A. In FIG. 3, the inside of the sweat collection device 1A is indicated by a broken line. The configuration and operation of the sweat collection device 1A will be described with reference to FIGS. 3 and 4.

The sweat collection device 1A of this embodiment is different from the sweat collection device 1 of the first embodiment in the configuration of a discharge section 12A. The other configuration is the same as that of the sweat collection device 1 of the first embodiment. The same reference numerals are given to the same components as those of the first embodiment. Further, the description of the same components will be omitted in the following description.

In the sweat collection device 1A of this embodiment, the discharge section 12A is not constituted by hole sections 122 (capillaries) in the first embodiment, but is constituted by a porous material to serve as a fine communication hole member. The porous material may be, for example, a porous glass, a cellulose fiber, or a cellulose particle, each having hydrophilicity. It may also a porous material such as a chemical fiber subjected to a hydrophilic treatment or a sponge subjected to a hydrophilic treatment. In this embodiment, the discharge section 12A is formed by placing a plate-shaped porous glass 123 inside the case of the discharge section 12A.

According to this configuration, when the sweat 8 flowing through the transfer section 11 has reached the discharge section 12A, the sweat 8 permeates the fine holes inside the porous glass 123, and is transferred to a terminal end portion of the discharge section 12A and discharged to the outside air. Incidentally, in FIGS. 3 and 4, the flow of the sweat 8 is schematically shown by the arrow.

According to this configuration, the sweat collection device 1A can promptly transfer the sweat 8 from the collection section 10 to the transfer section 11 and to the discharge section 12A by evaporating the sweat 8 at any time after completion of the measurement. Due to this, the sweat 8 immediately after excreted and collected by the collection section 10 can be promptly measured by the stimulus-responsive gel 21, and the sweat 8 after the measurement can be promptly transferred from the stimulus-responsive gel 21.

According to the above-mentioned embodiment, although the configuration is slightly different from that of the first embodiment, the same advantageous effects as those of the first embodiment can be obtained.

### Third Embodiment

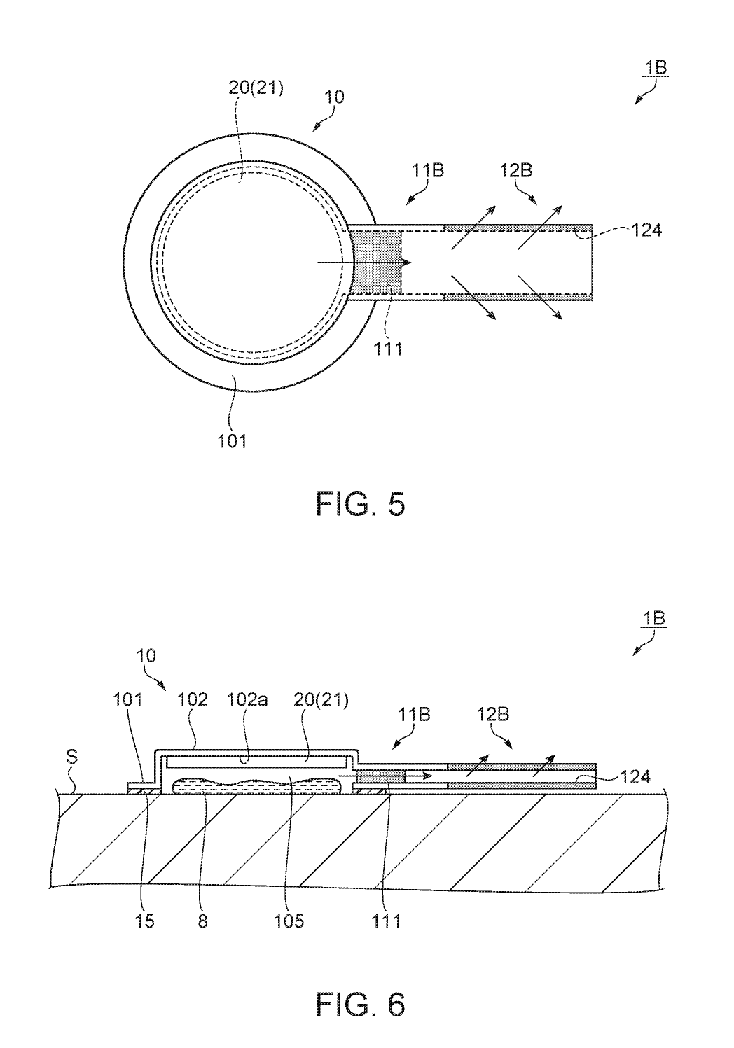

FIG. 5 is a plan view schematically showing a configuration of a sweat collection device 1B according to a third embodiment. FIG. 6 is a cross-sectional view schematically showing a configuration of the sweat collection device 1B. In FIG. 5, the inside of the sweat collection device 1B is indicated by a broken line. The configuration and operation of the sweat collection device 1B will be described with reference to FIGS. 5 and 6.

The sweat collection device 1B of this embodiment is different from the sweat collection device 1 of the first embodiment in the configuration of a transfer section 11B and a discharge section 12B. The other configuration is the same as that of the sweat collection device 1 of the first embodiment. The same reference numerals are given to the same components as those of the first embodiment. Further, the description of the same components will be omitted in the following description.

In the sweat collection device 1B of this embodiment, the discharge section 12B is not constituted by hole sections 122 (capillaries) in the first embodiment, but is constituted by a porous material in a cylindrical shape to serve as a fine communication hole member. Then, in this embodiment, as the discharge section 12B, a porous glass 124 in a cylindrical shape (in this embodiment, in a cylindrical shape with a rectangular cross section) is placed. Further, the transfer section 11B is configured such that, unlike the transfer section 11 which is hollow in the first embodiment, a porous glass 111 which is a porous material to serve as a fine communication hole member is placed inside the hollow (flow channel) with a rectangular cross section of the transfer section 11B as a filling member.

According to this configuration, the sweat 8 transferred from the collection section 10 to the transfer section 11B permeates the porous glass 111 placed in the transfer section 11B and is transferred to the discharge section 12B. The sweat 8 transferred to the discharge section 12B permeates the cylindrical porous glass 124 and is discharged to the outside air. Incidentally, in FIGS. 5 and 6, the flow of the sweat 8 is schematically shown by the arrow.

According to this configuration, the sweat collection device 1B can promptly transfer the sweat 8 from the collection section 10 to the transfer section 11B and to the discharge section 12B by evaporating the sweat 8 at any time after completion of the measurement. Due to this, the sweat 8 immediately after excreted and collected by the collection section 10 can be promptly measured by the stimulus-responsive gel 21, and the sweat 8 after the measurement can be promptly transferred from the stimulus-responsive gel 21.

According to the above-mentioned embodiment, although the configuration is slightly different from that of the first embodiment, the same advantageous effects as those of the first embodiment can be obtained, and also the following advantageous effects can be obtained.

According to the sweat collection device 1B of this embodiment, in the case where the discharge section 12B is constituted by a cylindrical fine communication hole member (porous glass 124), the porous glass 111 constituted by a fine communication hole member is placed in a flow channel (of the transfer section 11B) between the discharge section 12B and the collection section 10 as the filling member. Therefore, in the case where after the sweat collection device 1B is attached to the skin S of the arm, a movement such as shaking the arm is performed, since the discharge section 12B is formed into a cylindrical shape, the occurrence of air bubbles due to the penetration of outside air from the discharge section 12B into the collection section 10 can be prevented. Further, the penetration of dust or the like can also be prevented. In addition, the sweat 8 in the collection section 10 can be actively sucked through the filling member (porous glass 111) and made to flow to the discharge section 12B.

### Fourth Embodiment

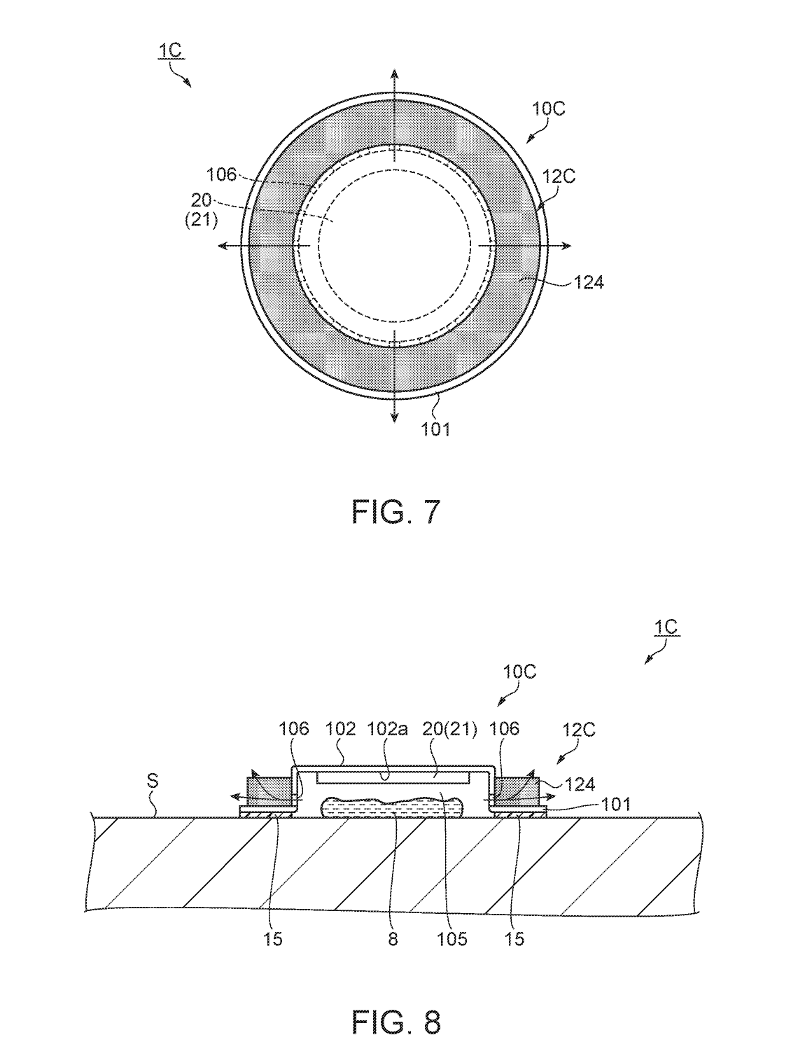

FIG. 7 is a plan view schematically showing a configuration of a sweat collection device 1C according to a fourth embodiment. FIG. 8 is a cross-sectional view schematically showing a configuration of the sweat collection device 1C. In FIG. 7, the inside of the sweat collection device 1C is indicated by a broken line. The configuration and operation of the sweat collection device 1C will be described with reference to FIGS. 7 and 8.

The sweat collection device 1C of this embodiment is configured to include a collection section 10C which collects sweat 8 and a discharge section 12C which discharges the sweat 8, and on the collection section 10C, the same response section 20 as in the first embodiment is placed. In this embodiment, unlike the sweat collection device 1 of the first embodiment, the discharge section 12C is continuous along an outer peripheral portion of the collection section 10C centering on the collection section 10C, and is formed throughout the entire outer periphery of the collection section 10C.

The collection section 10C is configured in substantially the same manner as the collection section 10 of the first embodiment. A different point is that hole sections 106 for allowing the sweat 8 collected by the collection section 10C to flow (transfer) to the discharge section 12C are formed in abase end portion from which the setting section 101 extends piercing therethrough throughout the entire periphery at a predetermined pitch.

The discharge section 12C is constituted by a porous material to serve as the fine communication hole member used in the discharge sections 12A and 12B in the second and third embodiments. The discharge section 12C of this embodiment is constituted by a porous glass 124 to serve as the porous material. More specifically, the discharge section 12C is formed into a doughnut-like flat plate shape along the outer periphery of the collection section 10C and is placed on the upper surface of the setting section 101. Incidentally, on the lower surface of the setting section 101, in the same manner as in the first embodiment, the adhesive member 15 is placed and is used when the sweat collection device 1C is attached to the surface of the skin S. The collection section 10C of the sweat collection device 1C is configured to be hermetically sealed by attaching the sweat collection device 1C to the skin S.

According to this configuration, the sweat 8 excreted from the skin S is accumulated in the space region 105 of the collection section 10C. The sweat 8 accumulated in the space region 105 comes into contact with the response section 20 (stimulus-responsive gel 21) placed on the upper part thereof and permeates the inside of the stimulus-responsive gel 21. Then, the stimulus-responsive gel 21 absorbs the sweat 8 and responds to (reacts with) lactic acid contained in the sweat 8.

In the case where the inside of the collection section 10C is filled with the sweat 8, the sweat 8 dissolved in the stimulus-responsive gel 21 or the sweat 8 accumulated in the space is pushed out to the discharge section 12C located on the side where the pressure of the sweat 8 is released through the respective hole sections 106. The sweat 8 transferred from the collection section 10C through the hole sections 106 comes into contact with the porous glass 124 constituting the discharge section 12C. Then, the sweat 8 permeates the fine holes inside the porous glass 124 and is transferred to the outer peripheral surface in contact with the outside air of the porous glass 124 and discharged to the outside air. Incidentally, in FIGS. 7 and 8, the flow of the sweat 8 is schematically shown by the arrow.

According to this configuration, the sweat collection device 1C can promptly transfer the sweat 8 from the collection section 10C to the discharge section 12C by evaporating the sweat 8 at any time after completion of the measurement. Due to this, the sweat 8 immediately after excreted and collected by the collection section 10C can be promptly measured by the stimulus-responsive gel 21, and the sweat 8 after the measurement can be promptly transferred from the stimulus-responsive gel 21.

According to the above-mentioned embodiment, although the configuration is slightly different from that of the first embodiment, the same advantageous effects as those of the first embodiment can be obtained, and also the following advantageous effects can be obtained.

According to the sweat collection device 1C of this embodiment, the discharge section 12C is configured to be continuous along an outer peripheral portion of the collection section 10C, and therefore, the sweat 8 collected by the collection section 10C can be made to more efficiently flow to the discharge section 12C and can be more efficiently discharged from the discharge section 12C to the outside as compared with the first embodiment.

### Fifth Embodiment

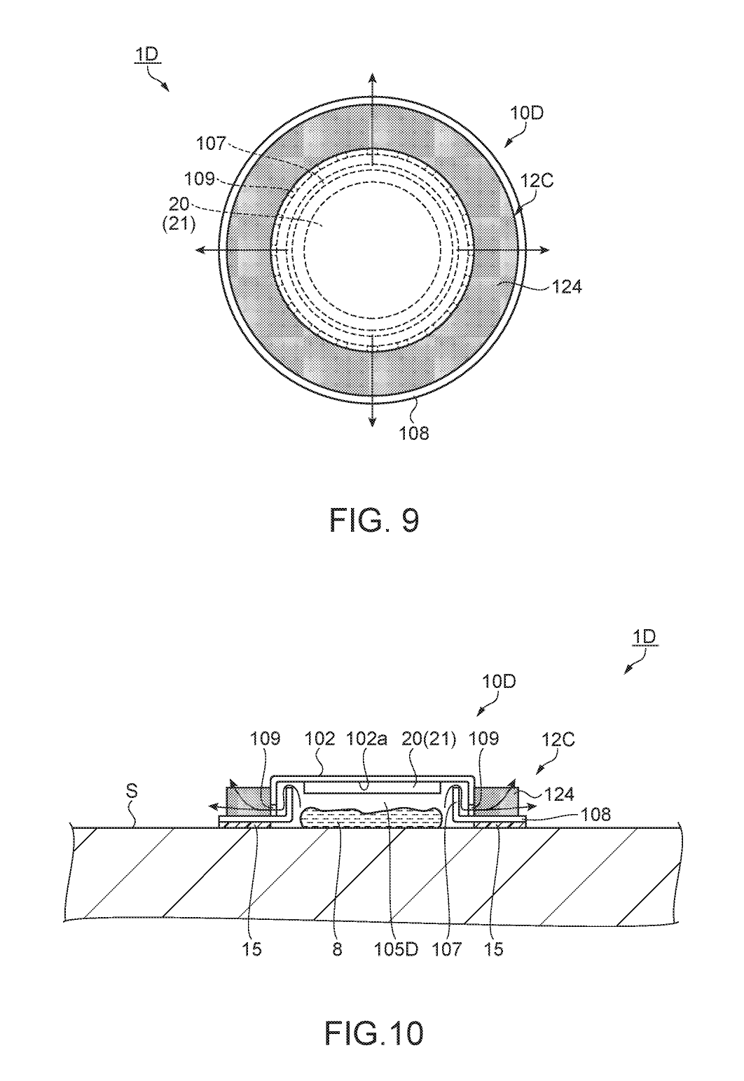

FIG. 9 is a plan view schematically showing a configuration of a sweat collection device 1D according to a fifth embodiment. FIG. 10 is a cross-sectional view schematically showing a configuration of the sweat collection device 1D. In FIG. 9, the inside of the sweat collection device 1D is indicated by a broken line. The configuration and operation of the sweat collection device 1D will be described with reference to FIGS. 9 and 10.

The sweat collection device 1D of this embodiment is different from the sweat collection device 1C of the fourth embodiment in the configuration of a collection section 10D. The other configuration is the same as that of the sweat collection device 1C of the fourth embodiment. The same reference numerals are given to the same components as those of the fourth embodiment. Further, the description of the same components will be omitted in the following description.

The sweat collection device 1D of this embodiment is configured to include a collection section 10D which collects sweat 8 and a discharge section 12C which discharges the sweat 8, and on the collection section 10D, the same response section 20 as in the fourth embodiment is placed. Unlike the collection section 10C of the fourth embodiment, the collection section 10D includes an accumulation section 107 for accumulating the sweat 8. The accumulation section 107 is formed in a cylindrical shape so as to surround the outer periphery of the response section 20.

Further, a flow section 108 in a doughnut-like flat plate shape extending from the outer periphery of the lower end portion to the outside is formed connected to the accumulation section 107. The flow section 108 has the function of the setting section 101 in the fourth embodiment, and in this embodiment, an adhesive member 15 is placed on the lower surface of the flow section 108, and a porous glass 124 is placed on the upper surface thereof as the discharge section 12C.

In the collection section 10D, a hole section 109 is formed in a place connected to the flow section 108 corresponding to the hole section 106 in the fourth embodiment. In the case where the collection section 10D is constituted by a synthetic resin material, first, the accumulation section 107 and the flow section 108 are integrally formed, and the collection section 10D in a concave shape remaining after forming the hole section 109 (in this case, it may be a notch) is formed. Then, this collection section 10D in a concave shape is fixed to the upper surface of the flow section 108, whereby the collection section 10D is formed. The fixing may be performed by bonding, welding, or the like.

Incidentally, by the adhesive member 15 placed on the lower surface of the flow section 108, the collection section 10D of the sweat collection device 1D is configured to be hermetically sealed by attaching the sweat collection device 1D to the surface of the skin S. Further, in this embodiment, a region surrounded by the response section 20, the accumulation section 107, and the skin S is formed as an accumulation region 105D corresponding to the space region 105 in the fourth embodiment.

According to this configuration, the sweat 8 excreted from the skin S is accumulated in the accumulation region 105D of the collection section 10D. The sweat 8 accumulated in the accumulation region 105D comes into contact with the response section 20 (stimulus-responsive gel 21) placed on the upper part thereof and permeates the inside of the stimulus-responsive gel 21. Then, the stimulus-responsive gel 21 absorbs the sweat 8 and responds to (reacts with) lactic acid contained in the sweat 8.

In the case where the inside of the accumulation region 105D is filled with the sweat 8, the sweat 8 dissolved in the stimulus-responsive gel 21 or the sweat 8 accumulated in the region goes over the cylindrical wall of the accumulation section 107 and is accumulated also outside the wall, and thus, the sweat 8 is accumulated in the entire inside of the collection section 10D. Then, the sweat 8 is pushed out to the discharge section 12C located on the side where the pressure of the sweat 8 is released through the respective hole sections 109. The operation thereafter is the same as in the fourth embodiment. Incidentally, in FIGS. 9 and 10, the flow of the sweat 8 is schematically shown by the arrow.

According to the above-mentioned embodiment, although the configuration is slightly different from those of the first embodiment and the fourth embodiment, the same advantageous effects as those of the first embodiment and the fourth embodiment can be obtained, and also the following advantageous effects can be obtained.

According to the sweat collection device 1D of this embodiment, the collection section 10D includes the accumulation section 107 which surrounds the response section 20 and accumulates the sweat 8, and therefore, the response section 20 can be made to reliably respond to the sweat 8. Further, the sweat 8 after the measurement can be made to sequentially flow to the discharge section 12C through the flow section 108. According to this, the balance between the response in the response section 20 and the discharge in the discharge section 12C can be adjusted.

### Sixth Embodiment

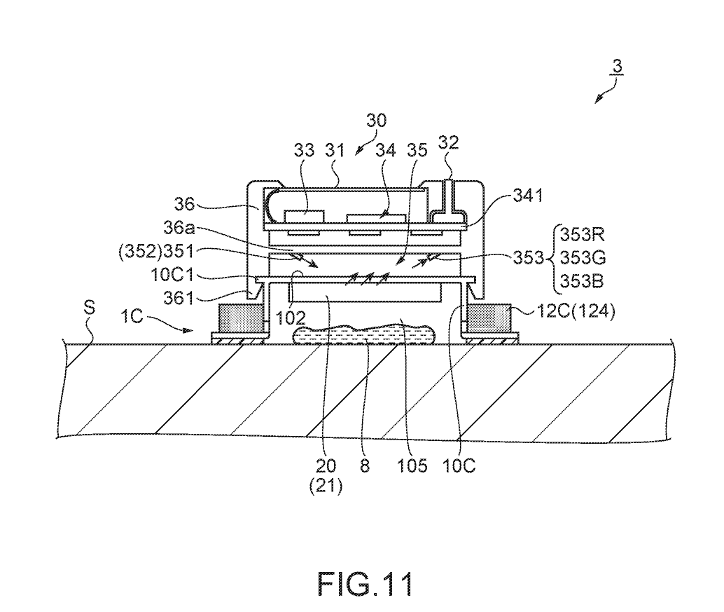

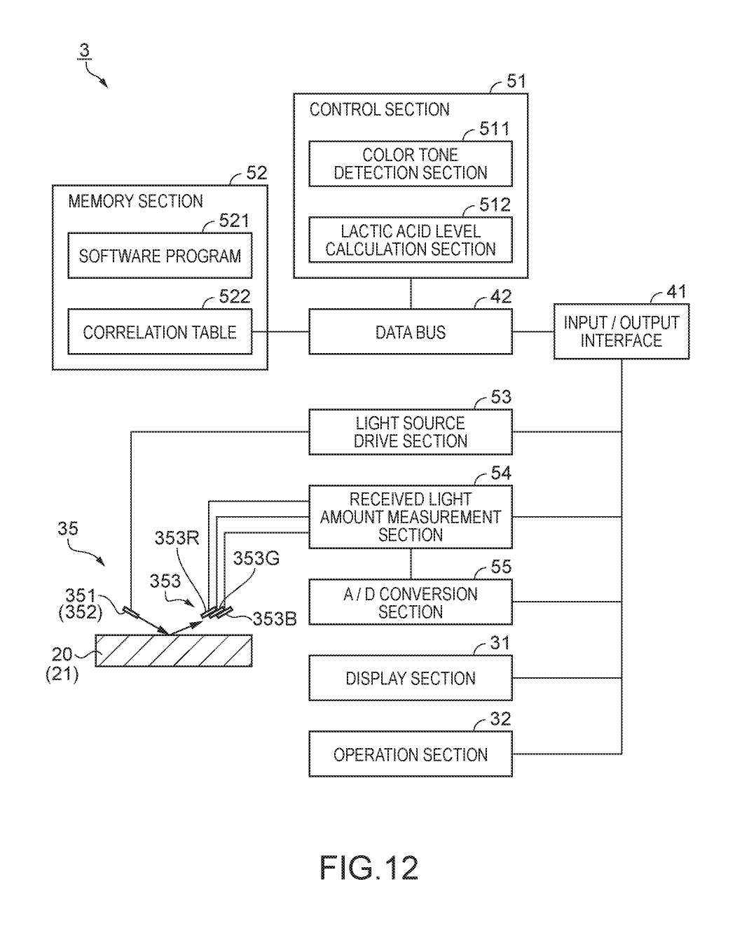

FIG. 11 is a cross-sectional view schematically showing a configuration of a sweat measurement device 3 according to a sixth embodiment. FIG. 12 is a view showing a schematic circuit configuration of the sweat measurement device 3. The configuration and operation of the sweat measurement device 3 as a measurement device will be described with reference to FIGS. 11 and 12.

The sweat measurement device 3 of this embodiment is constituted by using the sweat collection device 1C of the fourth embodiment. Incidentally, the sweat collection device 1C is a device capable of visually confirming the change in the color of the stimulus-responsive gel 21 which responds to lactic acid contained in sweat 8 through the transparent collection section 10C by a user. The sweat measurement device 3 of this embodiment is a device in which a lactic acid level is displayed on a display section 31 using an optical technique instead of the visual confirmation of the change in the color by a user.

The sweat measurement device 3 of this embodiment is configured to include the sweat collection device 1C and a device main body 30 detachably attached to the upper portion of the sweat collection device 1C. The device main body 30 is roughly constituted by a display section 31, an operation section 32, a power supply section 33, a circuit section 34, an optical detection section 35, and an outer packaging section 36 as a housing section for housing these members, and the like.

The outer packaging section 36 is formed into a substantially cylindrical shape. In the outer packaging section 36, a plurality of engaging sections 361 are formed on a lower end portion. Further, on an outer surface of a case of the collection section 10C of the sweat collection device 1C, a convex portion 10C1 is formed at a position corresponding to the engaging section 361. By pushing the outer packaging section 36 along a guiding section (not shown) from the upper part of the sweat collection device 1C so as to cover the collection section 10C, the engaging section 361 is engaged with the convex portion 10C1, whereby the outer packaging section 36 is fitted to the sweat collection device 1C. Incidentally, the fitting method is not limited to the engaging structure. As the fitting method, a method in which the outer packaging section 36 is adhered to an outer peripheral portion of the upper surface 102 of the collection section 10C with an adhesive member or the like may be adopted.

On a bottom surface portion 36a of the outer packaging section 36, the optical detection section 35 which optically detects the change in the color of the stimulus-responsive gel 21 located on the lower side of the bottom surface portion 36a is placed. On the upper part of the bottom surface portion 36a, the circuit section 34 is placed. The circuit section 34 includes a circuit board 341. In the circuit board 341, an electrical circuit system which drives and controls of the optical detection section 35 and the display section 31, and also constitutes a conversion section, a calculation section, or the like is placed. The electrical circuit system is constituted by a plurality of electrical elements. The conversion section converts a data signal detected by the optical detection section 35. Further, the calculation section calculates the amount of lactic acid contained in the sweat 8 based on the data signal.

The power supply section 33 constituted by a storage battery is placed side by side with the electrical circuit system constituting the circuit section 34. A power supply connector (not shown) for charging the storage battery is fixed in a state of being exposed from the outer packaging section 36. On the upper part of the circuit section 34, the display section 31 which displays a lactic acid level as the result of calculation by the calculation section and is constituted by, for example, a liquid crystal panel is placed. In the display section 31, other than the lactic acid level, a variety of information (a measurement state and the like) is displayed. In addition, on the upper part of the circuit section 34, the operation section 32 constituted by, for example, a key switch for giving a variety of instructions to the circuit section 34 is placed. In the instruction operation, an instruction of start/stop of measurement of a lactic acid level and the like are included.

The optical detection section 35 is configured to include a light source section 351 and a light receiving section 353. The light source section 351 includes a white light source 352 which emits white light to the stimulus-responsive gel 21. The white light source 352 is not particularly limited, but is constituted by, for example, a light-emitting diode (LED) or the like in this embodiment. The light receiving section 353 includes detection sensors, each of which detects reflection light to be reflected from the stimulus-responsive gel 21 when the stimulus-responsive gel 21 is irradiated with white light by the light source section 351 for each color light (red light, green light, and blue light). Each detection sensor is constituted by a phototransistor, a color filter, a condenser lens, and the like. Here, the detection sensor for red light is denoted by the reference numeral 353R, the detection sensor for green light is denoted by the reference numeral 353G, and the detection sensor for blue light is denoted by the reference numeral 353B.

Incidentally, the white light emitted from the light source section 351 (white light source 352) is incident from the upper surface 102 of the collection section 10C constituted by a transparent synthetic resin and is applied to the stimulus-responsive gel 21. Then, the reflection light which reflects the structural color of the stimulus-responsive gel 21 is emitted from the upper surface 102, and applied to the light receiving section 353 (detection sensors 353R, 353G, and 353B).

As shown in FIG. 12, the sweat measurement device 3 includes, as the electrical circuit system, a control section 51 which controls the entire operation of the sweat measurement device 3, a memory section 52 which stores a variety of information, a light source drive section 53, a received light amount measurement section 54, an A/D conversion section 55, the display section 31, and the operation section 32. The light source drive section 53, the received light amount measurement section 54, the A/D conversion section 55, the display section 31, and the operation section 32 are connected to the control section 51 through an input/output interface 41 and a data bus 42. Incidentally, the control section 51 is constituted by a central processing unit (CPU) which performs a variety of calculation processing as a processor, or the like.

The light source drive section 53 is connected to the light source section 351 and drives the white light source 352. The light source drive section 53 is connected to the control section 51 and controls the turning on and off of the white light source 352 and the amount of light according to the instruction from the control section 51.

The received light amount measurement section 54 is connected to the light receiving section 353 (the detection sensors 353R, 353G, and 353B for each color light). Then, the received light amount measurement section 54 drives the detection sensors 353R, 353G, and 353B.

The detection sensor 353R for red light outputs a signal showing the luminance of a red component of the reflection light reflected from the surface of the stimulus-responsive gel 21 to the received light amount measurement section 54. Similarly, the detection sensor 353G for green light outputs a signal showing the luminance of a green component of the reflection light reflected from the surface of the stimulus-responsive gel 21 to the received light amount measurement section 54, and the detection sensor 353B for blue light outputs a signal showing the luminance of a blue component of the reflection light reflected from the surface of the stimulus-responsive gel 21 to the received light amount measurement section 54.

The received light amount measurement section 54 receives the signal showing the luminance of each color light and outputs the signal to the A/D conversion section 55. The A/D conversion section 55 outputs data showing the luminance of each color light to the control section 51. The light receiving section 353, the received light amount measurement section 54, and the A/D conversion section 55 correspond to the conversion section.

In the memory section 52, a software program 521, a correlation table 522, and the like are stored. In the software program 521, a control procedure for the operation of the sweat measurement device 3 is written. In the correlation table 522, data showing a relationship between the color tone of the stimulus-responsive gel 21 and the lactic acid level are shown.

The control section 51 performs control so that the lactic acid level is measured according to the software program 521 stored in the memory section 52. More specifically, the control section 51 includes a color tone detection section 511. The color tone detection section 511 drives the light source drive section 53 and turns on the white light source 352. The received light amount measurement section 54 receives a luminance signal for each color light from the detection sensor 353R, 353G, or 353B for each color light and outputs the signal to the A/D conversion section 55. The A/D conversion section 55 outputs the luminance data of each color light to the control section 51. The color tone detection section 511 calculates the color tone of the stimulus-responsive gel 21 using the luminance data of each color light.

Further, the control section 51 includes a lactic acid level calculation section 512. The lactic acid level calculation section 512 receives the data of the color tone of the stimulus-responsive gel 21 from the color tone detection section 511, and calculates a lactic acid level with reference to the data of the color tone of the stimulus-responsive gel 21 and the correlation table 522. The control section 51 performs control so that the lactic acid level calculated by the lactic acid level calculation section 512 is output to the display section 31. Further, in this embodiment, the control section 51 sequentially calculates the lactic acid level which transitions, and displays a graph showing the transition of the lactic acid level on the display section 31. A user can confirm the lactic acid level and the transition of the lactic acid level by viewing the display section 31. Further, the control section 51 controls the operation of the sweat measurement device 3 according to the signal input from the operation section 32. Incidentally, the color tone detection section 511 and the lactic acid level calculation section 512 correspond to the calculation section.

According to the above-mentioned embodiment, the following advantageous effects are obtained.

The sweat measurement device 3 as the measurement device of this embodiment includes the sweat collection device 1C, the conversion section (the light receiving section 353, the received light amount measurement section 54, and the A/D conversion section 55) which converts the color of the structural color of the response section 20 (the stimulus-responsive gel 21) into a data signal and outputs the data signal. Further, the sweat measurement device 3 includes the calculation section (the color tone detection section 511 and the lactic acid level calculation section 512) which calculates the lactic acid level in the sweat 8 from the data signal output from the conversion section, the display section 31 which displays the result of calculation (lactic acid level) by the calculation section, and the outer packaging section 36 to serve as a housing section which houses the response section 20, the conversion section, the calculation section, and the display section 31. According to such a sweat measurement device 3, the amount of lactic acid contained in the sweat 8 can be continuously measured using a non-invasive technique and displayed on the display section 31. Therefore, by attaching this sweat measurement device 3 to, for example, an arm or the like and performing an exercise or the like, a user can easily confirm the change in the amount of lactic acid contained in the sweat 8 or the like during the exercise.

### Seventh Embodiment

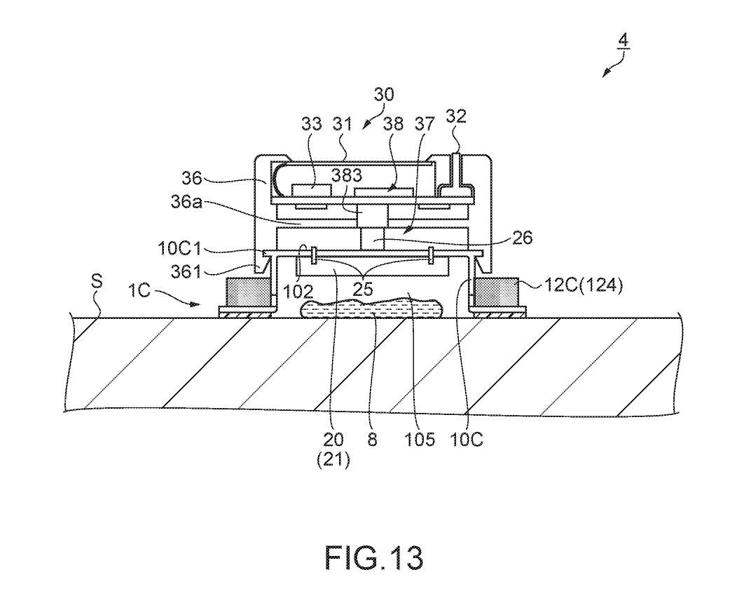

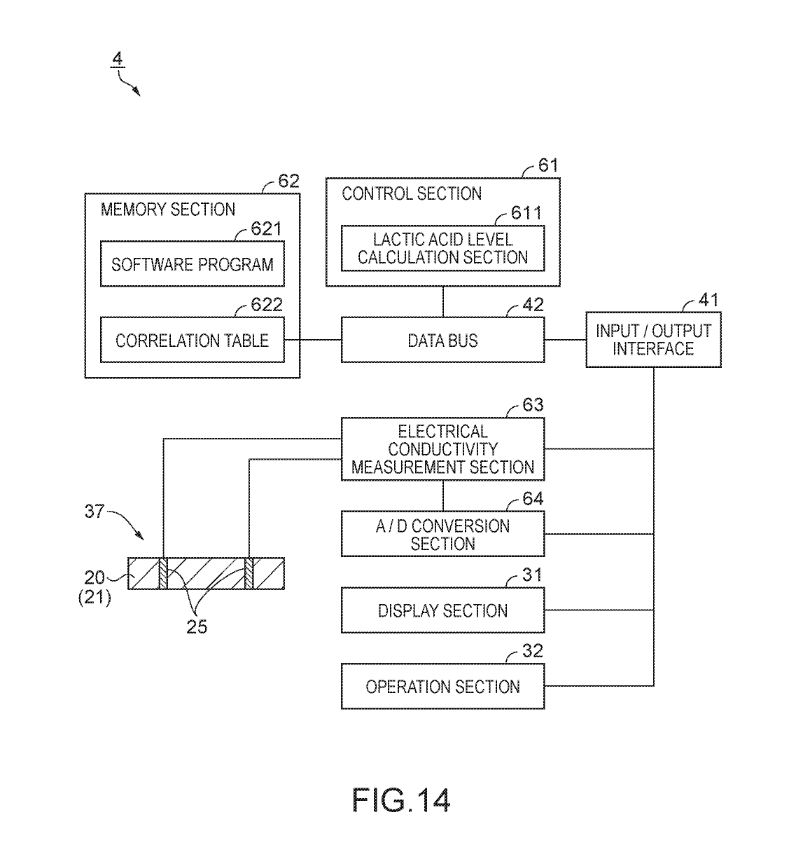

FIG. 13 is a cross-sectional view schematically showing a configuration of a sweat measurement device 4 according to a seventh embodiment. FIG. 14 is a view showing a schematic circuit configuration of the sweat measurement device 4. The configuration and operation of the sweat measurement device 4 as a measurement device will be described with reference to FIGS. 13 and 14. The description of the same configuration and operation as those in the sixth embodiment will be omitted in the following description.

While the sweat measurement device 3 of the sixth embodiment detects the change in the lactic acid level by the change in the structural color (color) of the stimulus-responsive gel 21, the sweat measurement device 4 of this embodiment detects the change in the lactic acid level by the change in the electrical conductivity of the stimulus-responsive gel 21, which is a different point from the sweat measurement device 3.

The stimulus-responsive gel 21 reacts with lactic acid and expands and contracts depending on the amount of lactic acid as described above. By changing the form of the stimulus-responsive gel 21 by expansion and contraction, the electrical conductivity of the stimulus-responsive gel 21 changes. Therefore, by placing a pair of electrodes 25 inside the stimulus-responsive gel 21 and measuring the resistance between the electrodes, the level of lactic acid permeating the inside of the stimulus-responsive gel 21 can be detected. The material of the electrodes 25 may be any as long as it is a material having electrical conductivity and corrosion resistance, and nickel- or gold-plated copper, or other than these, a metal such as platinum, gold, or silver, or carbon can be used.

The sweat measurement device 4 of this embodiment is configured in substantially the same manner as the sweat measurement device 3 of the sixth embodiment. What differs greatly is that the sweat measurement device 4 includes a resistance detection section 37 in place of the optical detection section 35. Accompanying this, the sweat measurement device 4 includes a circuit section 38 in place of the circuit section 34.

The sweat measurement device 4 includes the sweat collection device 1C in the same manner as in the sixth embodiment. However, the sweat collection device 1C is configured such that the pair of electrodes 25 to be inserted into the stimulus-responsive gel 21 from the upper surface 102 of the collection section 10C are placed. Further, on the upper surface 102, a connector 26 to be connected to a connector 383 which is placed in the circuit section 38 is placed. This connector 26 is electrically connected to the two electrodes 25 through a lead wire (not shown).

As shown in FIG. 14, the sweat measurement device 4 includes, as an electrical circuit system, a control section 61 which controls the entire operation of the sweat measurement device 4, a memory section 62 which stores a variety of information, an electrical conductivity measurement section 63, an A/D conversion section 64, a display section 31, and an operation section 32. The electrical conductivity measurement section 63, the A/D conversion section 64, the display section 31, and the operation section 32 are connected to the control section 61 through an input/output interface 41 and a data bus 42.

The electrical conductivity measurement section 63 applies a voltage to the pair of electrodes 25 placed in the stimulus-responsive gel 21 and detects an electrical current flowing between the electrodes 25. The electrical conductivity of the stimulus-responsive gel 21 changes depending on the lactic acid level in the stimulus-responsive gel 21. Therefore, by detecting an electrical current flowing through the stimulus-responsive gel 21 by the electrical conductivity measurement section 63, the electrical conductivity of the stimulus-responsive gel 21 corresponding to the lactic acid level can be detected.

The A/D conversion section 64 converts the electrical conductivity of the stimulus-responsive gel 21 detected by the electrical conductivity measurement section 63 into a digital data signal and outputs the data signal. The electrical conductivity measurement section 63 and the A/D conversion section 64 constitute a conversion section.

In the memory section 62, a software program 621, a correlation table 622, and the like are stored. In the software program 621, a control procedure for the operation of the sweat measurement device 4 is written. In the correlation table 622, data showing a relationship between the electrical conductivity of the stimulus-responsive gel 21 and the lactic acid level are shown.

The control section 61 performs control so that the lactic acid level is measured according to the software program 621 stored in the memory section 62. More specifically, the control section 61 includes a lactic acid level calculation section 611 as a calculation section. The lactic acid level calculation section 611 receives a data signal of the electrical conductivity from the A/D conversion section 64, and then, calculates a lactic acid level with reference to the value of the data signal and the correlation table 622.

In addition, the control section 61 controls the value of the voltage to be applied to the electrodes 25 by the electrical conductivity measurement section 63. Further, the control section 61 performs control so that the lactic acid level calculated by the lactic acid level calculation section 611 is output to the display section 31. Further, in this embodiment, the control section 61 sequentially calculates the lactic acid level which transitions, and displays a graph showing the transition of the lactic acid level on the display section 31. A user can confirm the lactic acid level and the transition of the lactic acid level by viewing the display section 31. Further, the control section 61 controls the operation of the sweat measurement device 4 according to the signal input from the operation section 32.

According to the above-mentioned embodiment, the following advantageous effects are obtained.

The sweat measurement device 4 as the measurement device of this embodiment includes the sweat collection device 1C, the conversion section (the electrical conductivity measurement section 63 and the A/D conversion section 64) which converts the electrical conductivity of the response section (the stimulus-responsive gel 21) into a data signal and outputs the data signal. Further, the sweat measurement device 4 includes the calculation section (the lactic acid level calculation section 611) which calculates the lactic acid level in sweat 8 from the data signal output from the conversion section, the display section 31 which displays the result of calculation (lactic acid level) by the calculation section, and the outer packaging section 36 to serve as a housing section which houses the response section 20, the conversion section, the calculation section, and the display section 31. According to such a sweat measurement device 4, the amount of lactic acid contained in the sweat 8 can be continuously measured using a non-invasive technique and displayed on the display section 31. Therefore, by attaching this sweat measurement device 4 to, for example, an arm or the like and performing an exercise or the like, a user can easily confirm the change in the amount of lactic acid contained in the sweat 8 or the like during the exercise.

The invention is not limited to the above-mentioned embodiments and may be taken into practice by adding various changes, modifications, etc. thereto without departing from the gist of the invention. Hereinafter, modification examples will be described.

The sweat collection device 1 of the first embodiment is configured such that the entire outer case is formed from a transparent synthetic resin material. However, the invention is not limited thereto, and it is not necessary that the entire outer case be transparent, but only a region in which the change in the color of the response section 20 can be confirmed may be transparent. This can also be applied to the second to fifth embodiments.

The sweat collection device 1 of the first embodiment is configured to include the transfer section 11, but may be configured to include the collection section 10 and the discharge section 12 without including the transfer section 11. This can also be applied to the second embodiment.

The sweat collection device 1 of the first embodiment is configured such that the response section 20 is placed on the collection section 10, however, the response section 20 may be placed on the transfer section 11 other than on the collection section 10. In such a configuration, by measuring the amount of lactic acid in the transfer section 11 in which the sweat 8 accumulated in the collection section 10 is transferred to the discharge section 12 all at once, the entire sweat 8 accumulated in the collection section 10 can be measured together averagely. In such a case, a configuration in which the response section is placed on the inner side of the upper surface of the transfer section 11, and a space region is provided on the lower side thereof may be adopted. According to this, the sweat 8 passes through the response section and the space region provided on the lower side of the response section and flows to the discharge section 12, and therefore, lactic acid can be continuously measured. This can also be applied to the second embodiment.

The sweat collection device 1 of the first embodiment is configured such that the response section 20 is placed on the collection section 10, however, the response section 20 may be placed on the discharge section 12 other than on the collection section 10. In such a case, the response section is placed on the inner side of the hole section 122 and a slit-shaped hole section may be formed.

The sweat collection device 1A of the second embodiment is configured such that the response section 20 is placed on the collection section 10, however, the response section 20 may be placed on the discharge section 12A other than on the collection section 10. In the case where the response section is placed on the discharge section 12A, when a chemical fiber (for example a non-woven fabric) subjected to a hydrophilic treatment is used as the porous material for the discharge section 12A, the discharge section 12A may be formed by immersing the non-woven fabric in a liquid obtained by mixing a polymer material, a solvent, and fine particles constituting the stimulus-responsive gel 21, and then irradiating the non-woven fabric with ultraviolet light in a state where the porosity is maintained. In this case, when the non-woven fabric is white, the visual recognizability of the change in the color of the stimulus-responsive gel 21 can be improved.

The sweat measurement device 3 of the sixth embodiment is constituted by using the sweat collection device 1C of the fourth embodiment. However, the invention is not limited thereto, and the sweat measurement device may be constituted by using the sweat collection device 1D of the fifth embodiment. This can also be applied to the seventh embodiment.

The sweat measurement device 3 of the sixth embodiment is configured such that a lactic acid level is displayed on the display section 31. However, a structural color may also be displayed along with a lactic acid level if the display section 31 enables color display.

The sweat collection device 1 of the first embodiment is configured such that one discharge section 12 is connected to the collection section 10 through one transfer section 11. However, the invention is not limited thereto, and a configuration in which a plurality of discharge sections 12 are placed in an outer peripheral portion of one collection section 10 although the transfer section 11 may not be connected may be adopted.

According to such a sweat collection device, a fluid collected by the collection section can be made to more efficiently flow to the discharge sections, and can be more efficiently discharged from the discharge sections to the outside.

The sweat collection device 1C of the fourth embodiment is configured such that the discharge section 12C is continuous along an outer peripheral portion of the collection section 10C centering on the collection section 10C, and is formed throughout the entire outer periphery of the collection section 10C. However, the invention is not limited thereto, and it is only necessary that the discharge section 12C be formed continuous along an outer peripheral portion of the collection section 10C, and it is not necessary that the discharge section 12C be formed throughout the entire outer periphery of the collection section 10C. This can also be applied to the fifth embodiment.

In the first to seventh embodiments, a lactic acid level in the sweat 8 is measured, however, a lactic acid level in another fluid excreted outside the body (such as tear, saliva, or urine), or a lactic acid level in blood may be measured. In any case, the measurement can be easily performed without using an enzyme.

By applying the first to seventh embodiments, in the medical field, for example, in the case where a patient's condition took a sudden turn for the worse or the like, the device can be used as a sensor which promptly measures the degree of increase in the lactic acid level in blood and tissues of the patient. In addition, the device can be used as a sensor which measures the increase in the lactic acid level in, for example, arteriosclerosis obliterans, decubitus ulcer, or the like.

By applying the first to seventh embodiments, in the food field, for example, by measuring the concentration of lactic acid during brewing or lactic acid fermentation, the state of progress of the respective steps can be confirmed. In addition, for example, as the evaluation of the quality of food, by measuring a lactic acid level, the freshness of the food can be confirmed.

By applying the first to seventh embodiments, in the pharmaceutical field, for example, the device can be used as a material for drug delivery targeting cancer tissues. In addition, the device can be used as a monitor for a bioreactor or a monitor for cell culture.

What is claimed is:

1. A fluid collection device, comprising:
a collection section which collects a fluid; and
a discharge section which discharges the fluid collected by the collection section from the collection section.

2. The fluid collection device according to claim 1, wherein
the fluid collection device is attached to an adherend, and
the collection section is configured to be hermetically sealed by attaching the fluid collection device to the adherend.

3. The fluid collection device according to claim 1, wherein the fluid collection device includes a transfer section which connects the collection section and the discharge section to each other.

4. The fluid collection device according to claim 3, wherein the fluid collection device includes a gel-like response section which is placed on any of the collection section, the transfer section, and the discharge section, and responds to a predetermined component contained in the fluid.

5. The fluid collection device according to claim 4, wherein any of the collection section, the transfer section, and the discharge section, on which the response section is placed, includes a region capable of visually recognizing the response section which responds to the predetermined component contained in the fluid.

6. The fluid collection device according to claim 4, wherein
the collection section includes a setting section to be set on the adherend, and
the setting section and the response section are spaced apart from each other.

7. The fluid collection device according to claim 3, wherein the surfaces coming into contact with the fluid of the collection section, the transfer section, and the discharge section have hydrophilicity.

8. The fluid collection device according to claim 1, wherein the discharge section is composed of a hole member constituted by a plurality of holes each of which is continuous with the outside of the fluid collection device from the collection section or the transfer section.

9. The fluid collection device according to claim 1, wherein the fluid collection device includes a plurality of discharge sections in an outer peripheral portion of the collection section.

10. The fluid collection device according to claim 1, wherein the discharge section is configured to be continuous along an outer peripheral portion of the collection section.

11. The fluid collection device according to claim 4, wherein the collection section includes an accumulation section which surrounds the response section and accumulates the fluid.

12. The fluid collection device according to claim 4, wherein the response section is configured to include a stimulus-responsive gel whose volume expands in response to the fluid.

13. The fluid collection device according to claim 2, wherein the fluid collection device includes an adhesive member for placing the collection section on the adherend.

14. A measurement device, comprising:
the fluid collection device according to claim 1;
a conversion section which converts a change in the response section into a data signal and outputs the data signal;
a calculation section which calculates the amount of the predetermined component contained in the fluid from the data signal output by the conversion section;
a display section which displays the result of calculation by the calculation section; and
a housing section which houses the response section, the conversion section, the calculation section, and the display section.

15. A measurement device, comprising:
the fluid collection device according to claim 2;
a conversion section which converts a change in the response section into a data signal and outputs the data signal;
a calculation section which calculates the amount of the predetermined component contained in the fluid from the data signal output by the conversion section;
a display section which displays the result of calculation by the calculation section; and
a housing section which houses the response section, the conversion section, the calculation section, and the display section.

16. A measurement device, comprising:
the fluid collection device according to claim 3;
a conversion section which converts a change in the response section into a data signal and outputs the data signal;
a calculation section which calculates the amount of the predetermined component contained in the fluid from the data signal output by the conversion section;
a display section which displays the result of calculation by the calculation section; and
a housing section which houses the response section, the conversion section, the calculation section, and the display section.

17. A measurement device, comprising:
the fluid collection device according to claim 4;
a conversion section which converts a change in the response section into a data signal and outputs the data signal;
a calculation section which calculates the amount of the predetermined component contained in the fluid from the data signal output by the conversion section;
a display section which displays the result of calculation by the calculation section; and
a housing section which houses the response section, the conversion section, the calculation section, and the display section.

18. A measurement device, comprising:
the fluid collection device according to claim 5;
a conversion section which converts a change in the response section into a data signal and outputs the data signal;
a calculation section which calculates the amount of the predetermined component contained in the fluid from the data signal output by the conversion section;
a display section which displays the result of calculation by the calculation section; and
a housing section which houses the response section, the conversion section, the calculation section, and the display section.

19. A measurement device, comprising:
the fluid collection device according to claim 6;
a conversion section which converts a change in the response section into a data signal and outputs the data signal;
a calculation section which calculates the amount of the predetermined component contained in the fluid from the data signal output by the conversion section;
a display section which displays the result of calculation by the calculation section; and
a housing section which houses the response section, the conversion section, the calculation section, and the display section.

20. A measurement device, comprising:
the fluid collection device according to claim 7;
a conversion section which converts a change in the response section into a data signal and outputs the data signal;
a calculation section which calculates the amount of the predetermined component contained in the fluid from the data signal output by the conversion section;
a display section which displays the result of calculation by the calculation section; and
a housing section which houses the response section, the conversion section, the calculation section, and the display section.
